(19) United States
(12) Patent Application Publication
(10) Pub. No.: US 2005/0080390 A1
(43) Pub. Date: Apr. 14, 2005

(54) Low-rise disposable undergarment

(76) Inventor: Shannon K. Stevens, New York, NY (US)

Correspondence Address: Shannon K. Stevens, Apt. 3WF, 506 East 84th Street, New York, NY, 10028, US

(21) Appl. No.: 10/931,127

(22) Filed: Aug. 30, 2004

Related U.S. Application Data
(60) Provisional application No. 60/498,464, filed on Aug. 28, 2003.

(51) Int. Cl. A61F 13/15

(52) U.S. Cl. 604/385.01

(57) ABSTRACT

This invention provides an article to be worn as an undergarment in low rise pants, said article having inside and outside surfaces and having front, center and back sections, said front section being substantially triangular in shape and being generally tapered from one end of the front section to the opposite end of the front section, said center section attached at the narrower end of said front section and attached at the opposite end to said back portion, said article further comprising an adhesive material situated on the outside surface of at least one of said front, center or back sections.

### RELATED APPLICATION DATA

This application claims the benefit of U.S. Provisional Application No. 60/498,464, filed Aug. 28, 2003.

### FIELD OF INVENTION

This invention is related to the field of clothing, in particular undergarments.

### BACKGROUND OF THE INVENTION

The rise of pants—and in particular jeans—which is typically defined as the measurement between the crotch seam and the waist line, is normally eight to nine inches. On so-called “low-rider” or “low rise” articles of clothing, the rise is typically less than 7 and one-half inches and can be five inches or less. Because the typical undergarment generally covers more surface area than that, most are detectable above the waist line in such low-rise attire. Panties either protrude out the top, the sides, or the back.

On solution to this problem has been so-called low-rider panties or low-rider thongs which are full fabric undergarments containing side material. However, although the side material on such items fit the body lower on the hips below the waist, they also cause bunching on the sides or hip-area if too large or cause unsightly skin bulges that can be seen through the clothing, if too small. In many cases, the sides of low-rider thongs and panties are still visible above the waist line of low rise pants.

Another solution has been to simply not wear undergarments. However, besides being unhygienic, this is also quite uncomfortable for women—especially in regard to jeans—as the denim crotch seam can cause irritation.

Sanitary pads or panty liners also aren't a good alternative, as they don't fit the garment or the area properly, don't adhere thoroughly to the crotch seam, and tend to bunch uncomfortably in the middle.

### SUMMARY OF THE INVENTION

As a solution to this increasingly common problem, this invention relates to a disposable, adhesive, flexible, low-rise undergarment article which is free of sides and can be adjusted by being cut to fit a rise ranging from five to seven and one-half inches.

The present invention provides a solution to the above-defined problem by providing a disposable, low-rise panty made of fabric and containing an adhesive material that allows for securing the panty in the crotch region of an article of clothing and designed in such a way as to provide the normal protection of a undergarment without being visible in low-rise pants.

Because the invention is adhesive and sticks to the inside of the low-rise article of clothing—so as not to protrude—it will also be disposable. However, instead of being made of paper or cellulose as most disposable panties are, this invention is comprised of fabric material as most other materials have been known to cause infection, irritation, or chaffing in the female genital area.

As a disposable garment, it can also be sized to fit each individual article of clothing it is to be worn with on a case-by-case basis. It will be typically available in small, medium, and large for different body types, but generally at a “rise” of seven and one-half inches or less. To fit a lower rise, the undergarment can presized to fit or can be cut with scissors by the wearer for a custom fit.

Without sides, the undergarment will not protrude from the clothing or cause unattractive bunching or lumps, whether in clothing or on skin. The thong shape of the undergarment will prohibit visible panty lines.

### DESCRIPTION OF THE INVENTION

This invention provides an article to be worn as an undergarment in low rise pants, said article having inside and outside surfaces and having front, center and back sections, said front section being substantially triangular in shape and being generally tapered from one end of the front section to the opposite end of the front section, said center section attached at the narrower end of said front section and attached at the opposite end to said back portion, said article further comprising an adhesive material situated on the outside surface of at least one of said front, center or back sections.

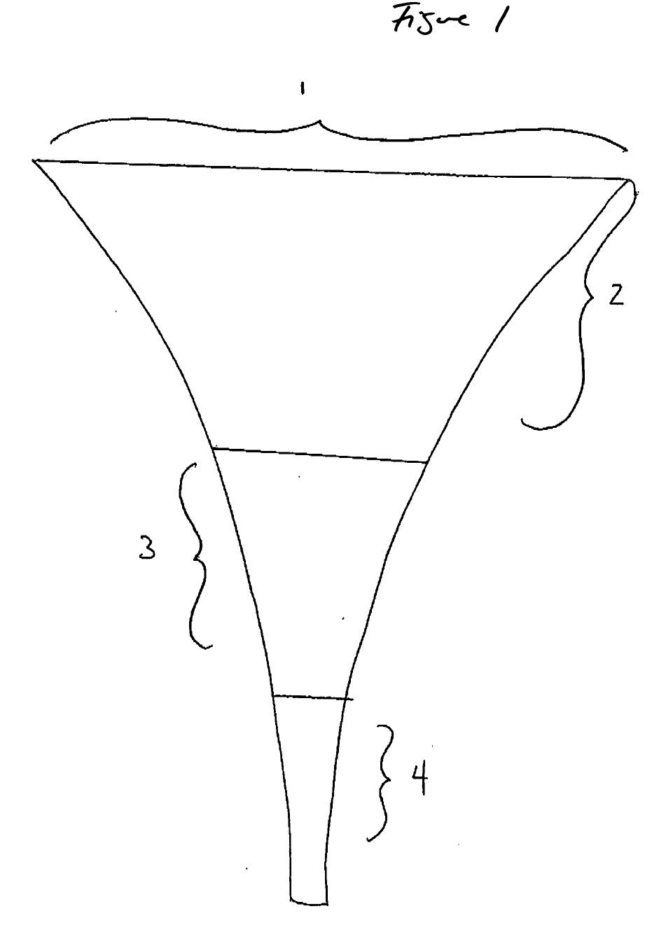

Referring to the embodiment depicted in FIG. 1, the article of the invention 1 comprises a front portion 2 that is generally triangular in shape, more particularly is shaped to fit the front panel of pants and to provide maximum coverage for the wearer. The center portion 3 will be generally triangular in shaped and preferably tapered along the sides, such that the top end of the center portion 3 that connects to front portion 2 will be as wider than the bottom end of center portion 3 that connects to the back portion 4. In a preferred embodiment, the center portion 3 further comprises additional material as a lining connected to the fabric of the article, the lining material preferably facing away from the outer garment and towards the wearer of the article. The back portion 4 will be generally rectangular shaped, such that it will lie along the crotch line of the garment to which the article of the invention is attached and will be of a length that is necessary to provide comfort to the wearer. In a separately preferred embodiment, the back portion 4 can also be a tapered generally triangular shape.

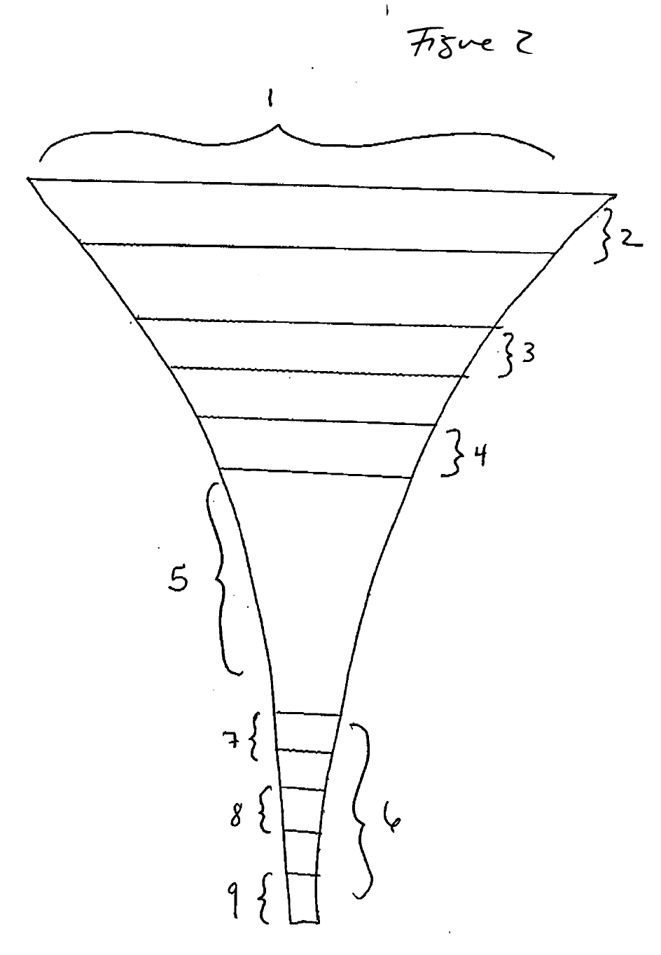

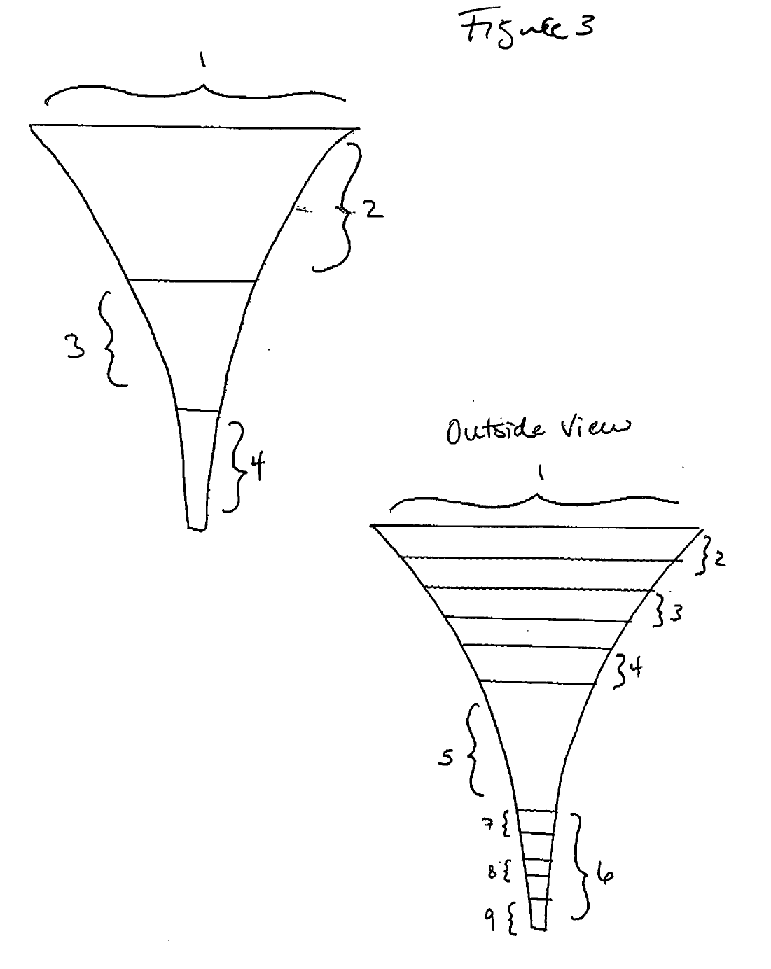

Referring to embodiments depicted in FIGS. 2 and 3, the article of the invention 1 comprises adhesive material applied to the outside of the front portion of the article, preferably arranged in strips (2, 3, 4) that are preferably arranged parallel to the top line of the front portion of the article 1 and perpendicular to the crotch line of the garment to which the article is to be attached. As used herein, the term “outside” of the article 1 is meant to refer to the side of the material that faces away from the wearer and towards the garment. Conversely, by the term “inside” of the article 1 is meant the side of the material that faces towards the wearer and away from the garment. In an additional embodiment, the article 1 can optionally comprise additional adhesive material applied to the back portion 6, optionally also applied in strips (7, 8, 9) arranged substantially parallel to the end of the back portion. In a preferred embodiment the middle portion 5 of article 1 does not contain any adhesive material. In separate embodiments (not shown) the adhesive material can also be applied in a single layer covering substantially the entire side of the article 1 facing the garment or in any pattern that allows for maintaining the article 1 releasably attached to the garment such that normal movement while wearing the garment does not detach the article 1 from the garment yet the article 1 is easily detached from the garment by hand when desired. The adhesive will typically be applied so that no rolling back of the outer corners of the article will occur and it will stay firmly in place inside the garment.

The article of the invention will preferably be made of a material that is a soft, flexible fabric, as opposed to paper or cellulose-based material, for comfort purposes. Such fabrics are generally known to those of ordinary skill in the art and comprise fabrics typically used for undergarment material and include, without limitation, such materials as cotton, polyesters, rayon and blends or combinations thereof. The adhesive useful in the article of the invention may be a pressure-sensitive, hot melt adhesive, Typically, a release paper will cover the adhesive prior to attaching the article to the garment. The low-rise disposable panty may be individually wrapped with a protective layer of plastic or similar material.

1. An article to be worn as an undergarment in low rise pants, said article having inside and outside surfaces and having front, center and back sections, said front section being substantially triangular in shape and being generally tapered from one end of the front section to the opposite end of the front section, said center section attached at the narrower end of said front section and attached at the opposite end to said back portion, said article further comprising an adhesive material situated on the outside surface of at least one of said front, center or back sections.
